(19) United States
(12) Plant Patent Application Publication
(10) Pub. No.: US 2013/0133115 P1
(43) Pub. Date: May 23, 2013

(54) Strawberry plant named 'Ginza'

(50) ;

(71) Applicant: John Larse, Aptos, CA (US)

(72) Inventor: John Larse, Aptos, CA (US)

(21) Appl. No.: 13/373,615

(22) Filed: Nov. 22, 2011

(51) Int. Cl. A01H 5/00

(52) U.S. Cl. PLT/208

(57) ABSTRACT

The present invention provides a new and distinct strawberry variety designated as ‘Ginza’ (a.k.a. ‘102011’).

### LATIN NAME OF THE GENUS AND SPECIES

Fragaria×ananassa

### VARIETAL DENOMINATION

‘Ginza’ (a.k.a. ‘102011’)

### BACKGROUND OF THE INVENTION

The present invention relates to a new and distinct day-neutral strawberry variety designated as ‘Ginza’ (a.k.a. ‘102011’). This new variety is the result of a controlled-cross between a female and a male parent cultivar (both unpatented, proprietary cultivars) made by the inventor and first fruited in Watsonville, Calif. growing fields.

Following selection and during testing, the plant was originally designated ‘102011’ and subsequently named ‘Ginza’. The new variety of ‘Ginza’ was asexually reproduced via runners (stolons) by the inventor in Watsonville, Calif. Asexual propagules from the original source have been tested in Watsonville growing fields and to a limited extent, grower fields in high elevation. The properties of this variety were found to be transmissible by such asexual reproduction. The cultivar is stable and reproduces true to type in successive generations of asexual reproduction.

### BRIEF SUMMARY OF THE INVENTION

This invention relates to a new and distinctive day-neutral type cultivar designated as ‘Ginza’. It is primarily adapted to the climate and growing conditions of the central coast of California. This region provides the necessary temperatures required for it to produce a strong vigorous plant and to remain in fruit production from late-April through October. The nearby Pacific Ocean provides the needed humidity and moderate day temperatures and evening chilling to maintain fruit quality for the production months.

The following traits in combination distinguish strawberry variety ‘Ginza’ from the known strawberry varieties. Plants for the botanical measurements in the present application were grown as annuals. In the following description, color references are made to The Royal Horticultural Society Colour Chart, 1995 Edition, except where general terms of ordinary dictionary significance are used.

The produced fruit is attractive and of excellent quality. Produced fruit is orange-red to medium-red at maturity, fruits are uniformly medium in size, firm, conical to oblate in shape with a smooth surface lacking creases. Seeds are well above the surface.

‘Ginza’ is primarily adapted to the growing conditions of the central coast of California, characterized by foliage small to medium in size and light to medium green in color. Production generally begins in April and produces continually into October with 40% to 50% of its production in the months of August through September.

‘Ginza ’ is different from strawberry variety ‘Bob ’, a.k.a. ‘105626’ and strawberry variety ‘Zarina’, a.k.a. ‘105218’ disclosed in the co-pending applications. The table below shows differences among these varieties.

| Variety | ‘Bob’ | ‘Ginza’ | ‘Zarina’ |
|---|---|---|---|
| Fruit Color at | light-red to | orange-red to | pink-red to |
| Maturity | medium-red | medium-red | light-red |
| Fruit Size | medium to large | uniformly | medium to |
| | | medium in size | large |
| Fruit Firmness | very firm | firm | firm |
| Fruit shape | conical to | conical to oblate | conical to |
| | cylindrical | | cylindrical |
| Surface Texture | smooth, lacking | smooth, lacking | smooth, lacking |
| | creases | creases | creases |
| Seed/Surface | Seeds are slightly | Seeds are well | Seeds may be |
| Position | sunken below the | above the | slightly sunken |
| | surface | surface | below the surface |

### DESCRIPTION OF THE DRAWINGS

The accompanying color photographs depict various characteristics of the ‘Ginza’ cultivar at various stages of development as nearly true as possible to make color reproductions.

FIG. 1 shows the plant in its production stage bearing fruit.

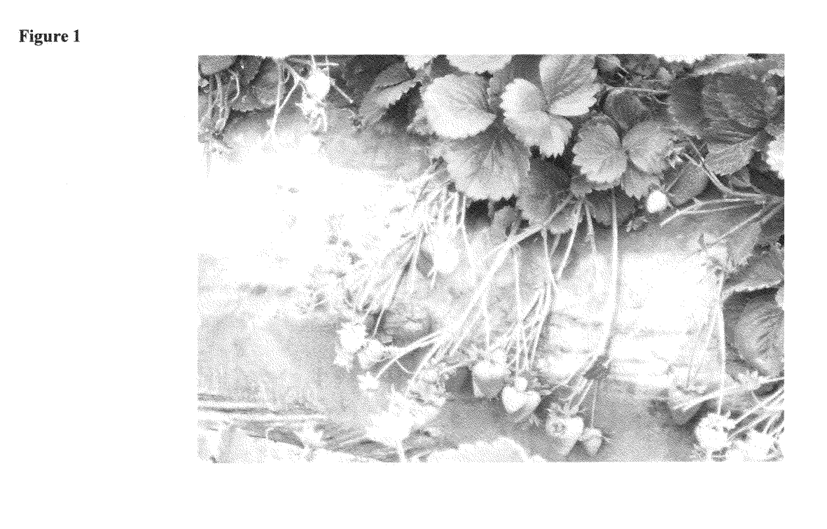

FIG. 2 shows the plant with flower and runners.

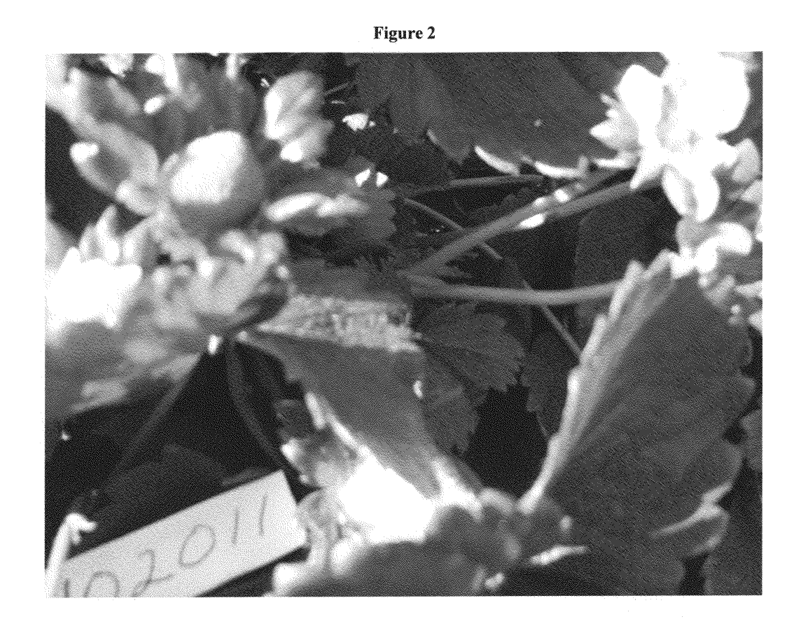

FIG. 3 shows typical leaves in the production cycle.

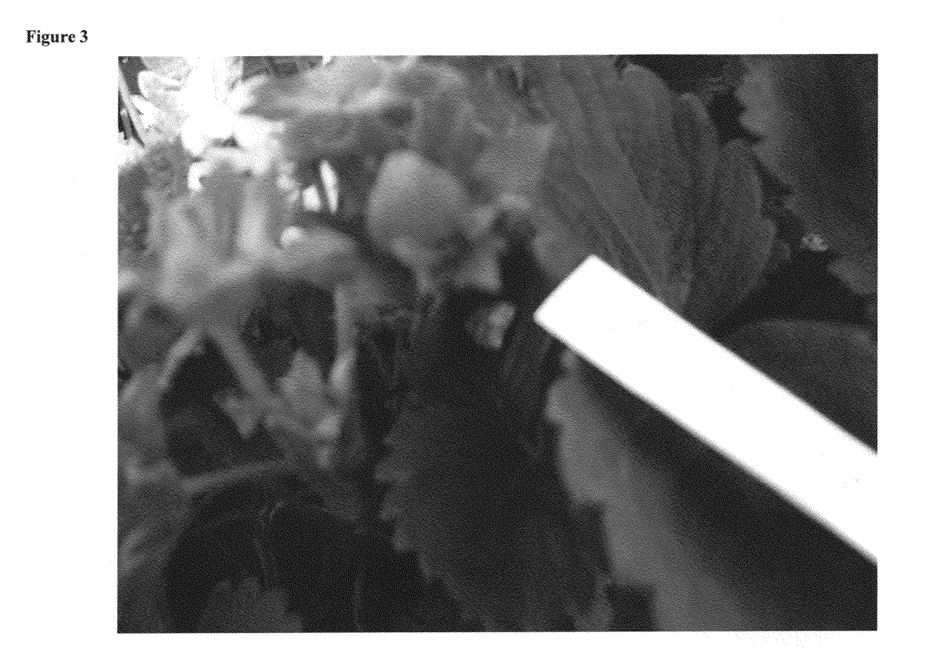

FIG. 4 shows ripe ‘Ginza’ fruit (October).

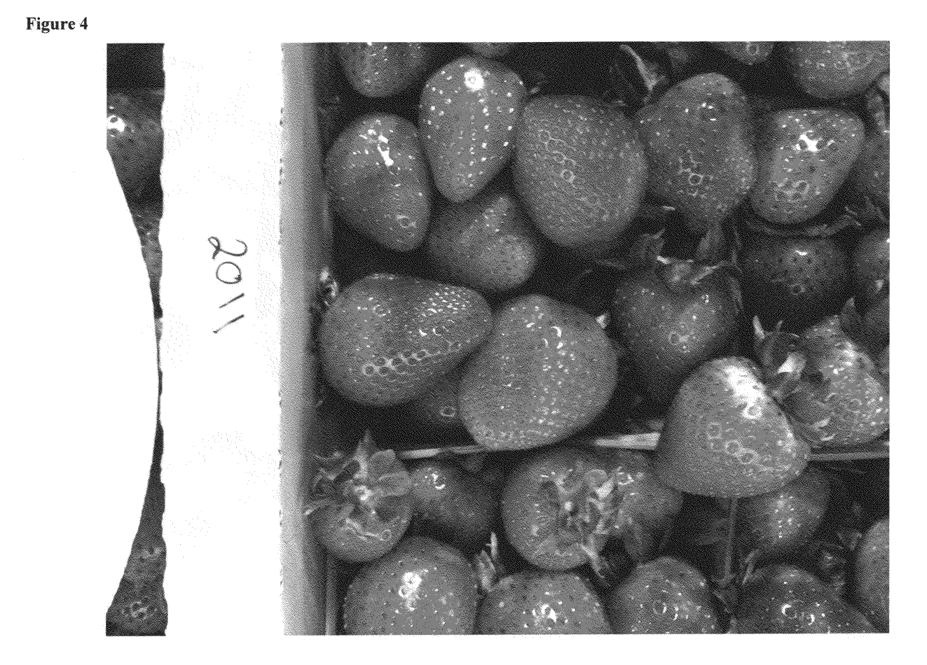

FIG. 5 shows ripe ‘Ginza’ fruit (June).

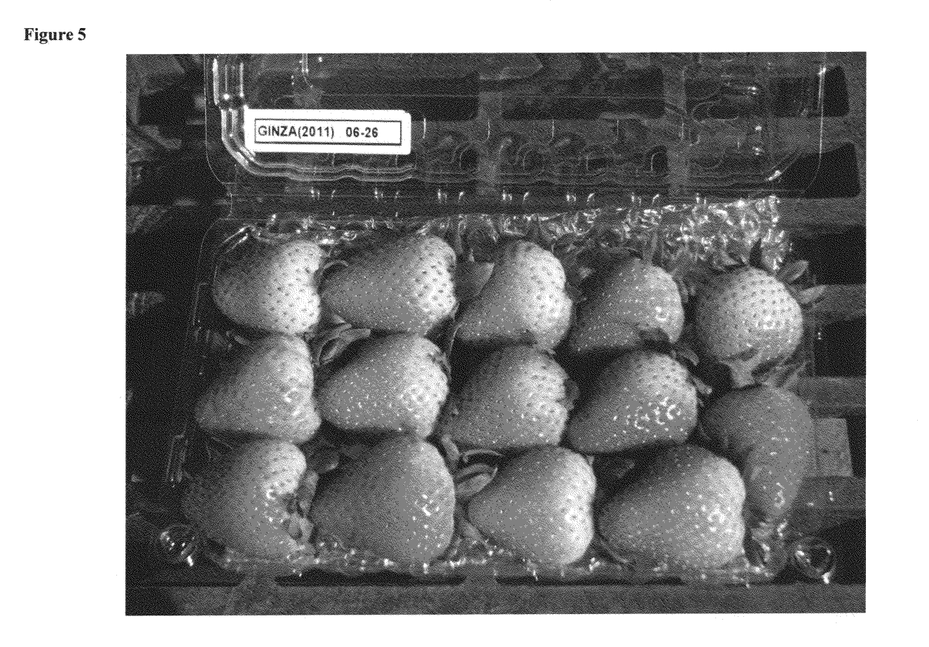

FIG. 6 shows sliced ‘Ginza’ fruit.

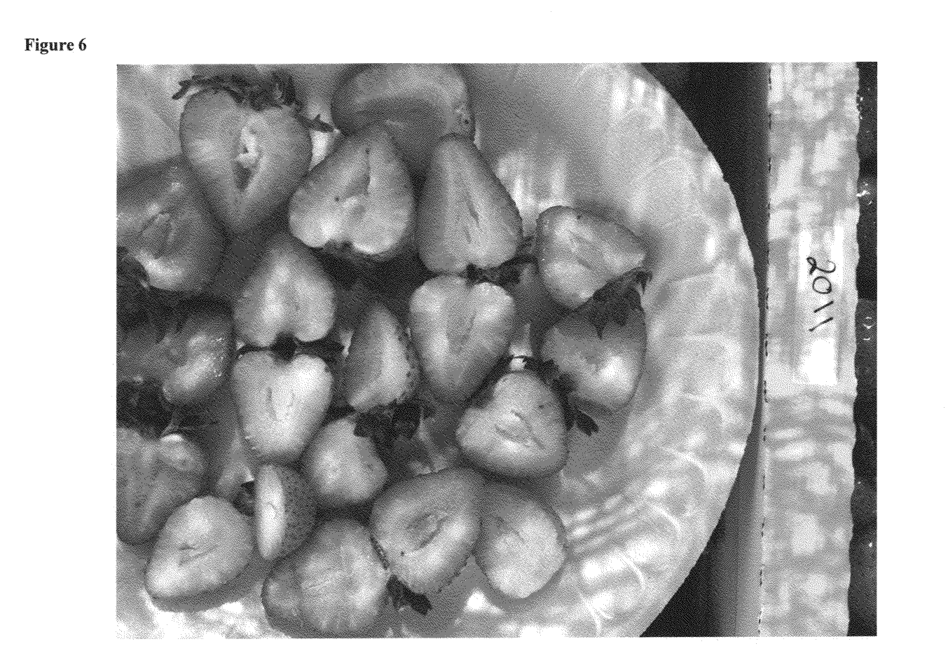

FIG. 7 shows ripe and near-ripe ‘Ginza’ fruits.

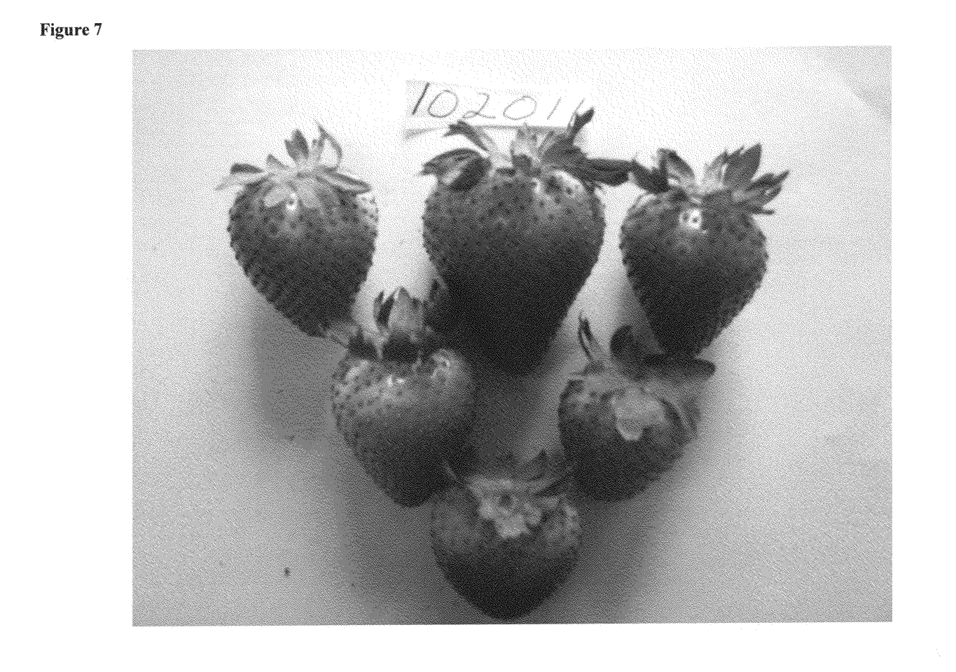

### DETAILED BOTANICAL DESCRIPTION

This invention relates to a new and distinctive day-neutral (ever-bearing) type cultivar designated as ‘Ginza’, but may be considered a weak day-neutral in that it is somewhat similar to a short day. ‘Ginza’ is typical of day-neutral strawberry cultivars and produces fruit regardless of the day length when treated appropriately in moderate, coastal climates. ‘Ginza's’ fruit is uniformly medium in size, that is medium-firm to firm and conical to cylindrical or conical to oblate in shape with seeds above the surface; color is light orange or orange-red to medium red at maturity.

Among the characteristics that distinguish the new variety from the other closely related varieties are the timing of fruiting, plant vigor, position of the inflorescences, leaf morphology, leaf size, leaf density, leaf color, and disease and pest resistance.

‘Ginza’ has not been observed under all possible environmental conditions, and the phenotype may vary significantly with variations in environment. The following observations, measurements, and comparisons describe this plant as grown at California, when grown in the field, unless otherwise noted. As stated above, the color determination is in accordance with The Royal Horticultural Society Colour Chart, 1995 Edition, except where general color terms of ordinary dictionary significance are used. Plants for the botanical measurements in the present application are annual plants.
Botanical classification: ‘Ginza’ is a fertile hybrid derived from a cross.
Common name: garden strawberry
General description:
   Plant habit.—moderate, coastal climates.
   Flowering, fruiting and production characteristics.—as typical of the species, the flowers are perfect, petals are pure white and rounded and entire; and anthers and receptacle are bright yellow. The calyx position is generally centered with 7 to 9 sepals that are light to medium green in color. The sepals are comprised of a single layer.
Foliar characteristics for ‘Ginza’: Plant size is short to medium in stature. The plant height ranges from 240-255 mm. The shape of the base is obtuse and light to medium in density. Average Crown per mature plant is 4 with a range of 3-4. Foliar color is medium to dark green on the adaxial surface of the leaf The abaxial surface of the leaf is light, silver-green color. No bract leaves were observed in samples. The shape of the leaf is almost circular with the width slightly exceeding the length. Each leaflet has three leaves. The leaves are moderately too highly glossy. The shape of the leaves is slightly concave to almost flat. There is minimal tertiary fruit grown from the variety.
Flower and fruit characteristics: The average number of petals per flower is 6 with a range of 5-7 petals. Average petal length is 11 mm with a range of 10-12 mm. The positioning of the inflorescence is predominately even with or above foliage. The flowers are mostly medium small to medium in size. The plant produces mostly conical fruit with some long cones as well as long wedges and the fruit color is dull to light gloss. The fruit size is medium to large with an average length of 50 mm, with a range of 31-79 mm, and width of 44 mm, with a range of 20-58 mm. The average length to width ratio of the fruit is about 1.21 with a range of 0.9-1.8. Seed position on fruit is somewhat indented. Fruit hardness was measured with a force dial and averaged 3.8 N to pierce the fruit 7.0 mm. The fruit's flavor is consistently good throughout the season.
Variety identification testing for ‘Ginza’: No matches to existing cultivars were found using SSR marker testing in a commercial database search.

1. A new and distinct cultivar of strawberry plant named ‘Ginza’ substantially as shown and described herein.
